(19) United States
(12) Patent Application Publication
(10) Pub. No.: US 2022/0160369 A1
(43) Pub. Date: May 26, 2022

(54) BILATERAL REVERSIBLE VENOUS TOURNIQUET BANDS

(71) Applicant: IPC, LLC DBA PlatinumCode, Lakeville, MN (US)

(72) Inventors: Martin J. Griffith, Prior Lake, MN (US); Ronald E. Griffith, Lakeville, MN (US); Stephen L. Griffith, Lakeville, MN (US)

(21) Appl. No.: 17/101,003

(22) Filed: Nov. 23, 2020

(51) Int. Cl. A61B 17/132

(52) U.S. Cl. CPC A61B 17/1322; A61B 2017/00955

(57) ABSTRACT

Disclosed herein are embodiments for a bilateral reversible disposable tourniquet band. The band tourniquet comprises an elongated member having a flat structure made of a thermoplastic elastomer material. The band has a first surface having a smooth texture, and a second surface having a non-smooth texture comprising a knurled pattern. The elongated member may have a thickness between 0.5 mm and 2 mm.

### BACKGROUND

### Technical Field

Embodiments generally relate to disposable venous tourniquet bands.

### Background

Healthcare professionals use disposable venous tourniquet bands to perform venipunctures and other procedures involving intravenous access to veins. In general, a tourniquet is used to apply pressure to a limb or extremity to limit blood flow. Disposable tourniquets are typically used for drawing blood from patients in a healthcare setting. Using the disposable variety of tourniquets is more hygienic and convenient than having to sterilize reusable tourniquets.

However, current disposable venous tourniquet bands are sometimes unsuitable or unpleasant for patients and healthcare providers due to, for example, physical limitations, skin conditions, technical preferences, etc.

### BRIEF DESCRIPTION OF THE DRAWINGS

The accompanying drawings are incorporated herein and form a part of the specification.

FIG. 1 illustrates a bilateral reversible disposable venous tourniquet band with textured and smooth sides, according to an example embodiment.

FIG. 2 illustrates a close-up view of a bilateral reversible disposable venous tourniquet band with textured and smooth sides, according to an example embodiment.

FIG. 3 illustrates a bilateral reversible disposable venous tourniquet band with textured and smooth sides in a large roll configuration, according to an example embodiment.

### DETAILED DESCRIPTION

Provided herein are embodiments for a bilateral reversible disposable venous tourniquet band with textured and smooth sides. Previous disposable venous tourniquet bands do not allow for accommodation based on possible physical requirements or limitations of patient skin as well as to provide for various degrees of relief and well-being and/or preference.

Embodiments of the bilateral reversible venous tourniquet band allow healthcare professionals to perform venipunctures and other procedures involving intravenous access to veins. The new tourniquet system provides options not previously available to the healthcare professional in one holistic system. Namely, the system may be used with the untextured/smooth side in contact with a patient's epidermis, or alternatively, as the situation may dictate, the band can be used with the textured side placed against the patient's skin.

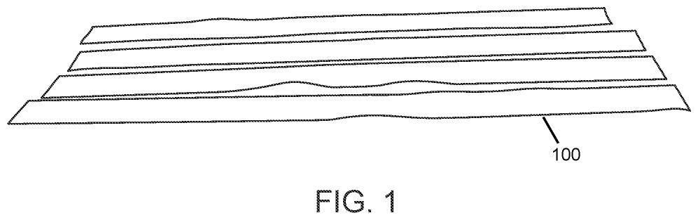

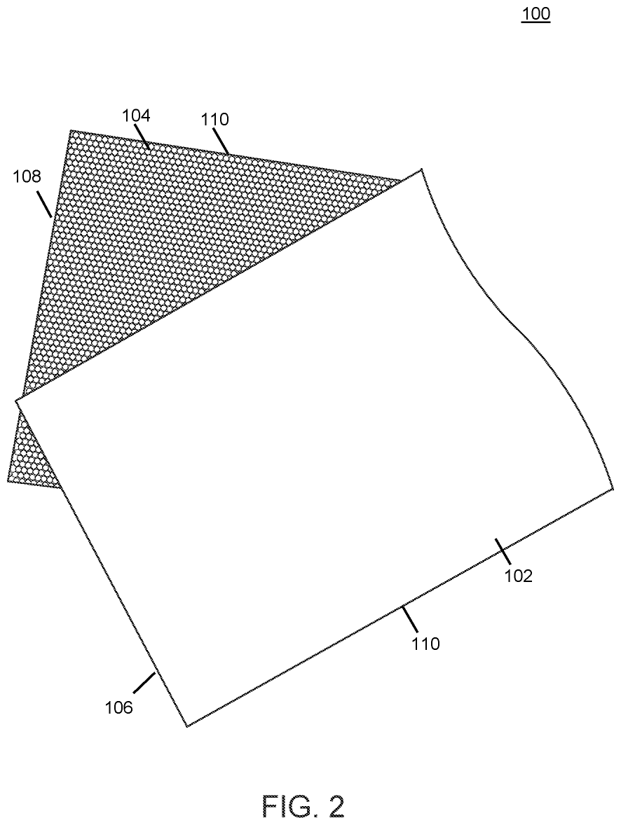

FIGS. 1 and 2 illustrate a bilateral reversible disposable tourniquet band 100, according to an example embodiment. Bilateral reversible venous tourniquet band 100 is comprised of an elongated member forming a disposable venous tourniquet having an outer surface (102), an inner surface (104) opposite of the outer surface, a first end (106) and a second end (108) opposite of the first end. The elongated member is comprised of a flat structure that has a width, a length and a thickness that may vary. The elongated member may be comprised of a flexible and elastic base material such as a thermoplastic elastomer. The elongated member may be comprised of an elastic material to allow for resilient stretching of the elongated strip. The elongated member may be comprised of a disposable material.

Regardless of the structure of the elongated member, the elongated member preferably includes a first end (106) and a second end (108) opposite of the first end as illustrated in FIG. 2. The elongated member further includes a first surface (102) (e.g. exterior surface) and a second surface (104) (e.g. interior surface) opposite of the first surface. This invention produces a first surface (102) and the second surface (104) of different textures one from the other. One of the surfaces (102) has a texture that is smooth while the opposite surface (104) has a non-smooth texture with a knurled pattern. The elongated member preferably has side edges (110) that are of equal length and are spaced apart consistently that extend from the first end (106) to the second end (108).

The knurled pattern of surface 104 may be any suitable pattern providing a gripping texture. In one example, the knurled pattern may be a crossed pattern with a pitch (distance between protrusions) between 0.1 mm and 1 mm.

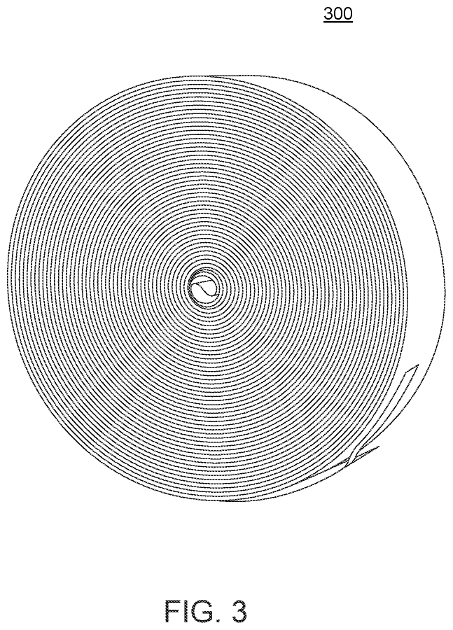

Tourniquet band 100 may have any suitable length and width for disposable use in a clinical setting. In particular embodiments, tourniquet band 100 may have any thickness between approximately 0.5 mm and 2 mm, and a length of approximately 18 inches. Tourniquet band 100 may be configured in any suitable manner for transportation and storage. For example, multiple tourniquet band 100 may be laid flat for transportation in a box, or rolled into individual reels. In particular embodiments, tourniquet band 100 may have a length of several feet and may be configured in a large roll that can be cut to a desired size at the time of use, as illustrated in FIG. 3.

Tourniquet band 100 may further include a colorant added to the base material to provide any desired color (e.g. white, violet, blue, green, yellow, red, orange, pink, phosphorescent).

It is to be appreciated that the Detailed Description section, and not the Summary and Abstract sections (if any), is intended to be used to interpret the claims. The Summary and Abstract sections (if any) may set forth one or more but not all exemplary embodiments of the invention as contemplated by the inventor(s), and thus, are not intended to limit the invention or the appended claims in any way.

While the invention has been described herein with reference to exemplary embodiments for exemplary fields and applications, it should be understood that the invention is not limited thereto. Other embodiments and modifications thereto are possible, and are within the scope and spirit of the invention. Further, embodiments (whether or not explicitly described herein) have significant utility to fields and applications beyond the examples described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

The breadth and scope of the invention should not be limited by any of the above-described exemplary embodiments, but should be defined only in accordance with the following claims and their equivalents.

What is claimed is:

1. A bilateral reversible disposable tourniquet band comprising:
an elongated member having a flat structure, the elongated member comprised of a thermoplastic elastomer material, the elongated member comprising:
a first surface having a smooth texture, and
a second surface having a non-smooth texture comprising a knurled pattern, the second surface being opposite the first surface,
wherein the elongated member has a thickness between 0.5 mm and 2 mm.

2. The bilateral reversible disposable tourniquet band of claim 1, wherein the knurled pattern comprises a crossed pattern with a pitch between *** mm and *** mm.

3. The bilateral reversible disposable tourniquet band of claim 1, wherein the band is packaged as a reel.

4. The bilateral reversible disposable tourniquet band of claim 1, wherein the band is packaged flat.

5. The bilateral reversible disposable tourniquet band of claim 1, wherein the band has a length of approximately 18 inches.

6. The bilateral reversible disposable tourniquet band of claim 1, wherein the band includes a colorant added to the thermoplastic elastomer material.
